(19) United States
(12) Patent Application Publication
(10) Pub. No.: US 2012/0128607 A1
(43) Pub. Date: May 24, 2012

(54) EXTRACT OF WHOLE SEEDS OF MORNINGA SP, AND USE THEREOF IN COSMETIC AND/OR DERMATOLOGICAL COMPOSITIONS

(75) Inventors: Anne Mandeau, Toulouse (FR); Helene Duplan, Auzeville Tolosan (FR)

(73) Assignee: PIERRE FABRE DERMO-COSMETIQUE, BOULOGNE (FR)

(21) Appl. No.: 13/377,943

(22) PCT Filed: Jun. 17, 2010
(86) PCT No.: PCT/FR2010/051207

§ 371 Date: Feb. 8, 2012

(30) Foreign Application Priority Data
Jun. 22, 2009 (FR) 0954235

(51) Int. Cl. A61K 8/97; A61Q 17/04; A61Q 19/02; A61Q 19/08

(52) U.S. Cl. 424/59; 424/769; 424/62

(57) ABSTRACT

The present invention relates to an extract of whole seeds of Moringa sp. containing oil (including triglycerides, fatty acids, and polar lipids) and polyphenols and to the use thereof in cosmetic and/or dermatological compositions.

The present invention relates to an extract of whole seeds of Moringa sp. containing oil (including triglycerides, fatty acids, and polar lipids) and polyphenols and to the use thereof in cosmetic and/or dermatological compositions.

The Moringa is a small tree indigenous to India, but cultivated everywhere in the world and naturalized in many environments where it is very popular. There are 13 species of Moringa belonging to the Moringaceae family, M. oleifera (synonym: Moringa pterygosperma) being the most well-known.

The Moringa oleifera is a small tree, 4 to 8 meters in height, with an open crown spread as an umbrella. The leaves are deciduous, 30 to 70 n cm long, the flowers are white and very fragrant, the fruit is a linear, elongated capsule, trigonal, coriaceous, and pendulous, with a length reaching 30 to 40 cm. The seeds are rounded trigons, 1.2 cm long and 1 cm wide, with three membranous wings extending from the seeds and 2 cm long.

Wild or cultivated in a tropical, humid, or very dry climate, the tree can survive under extreme conditions and grow very rapidly. Its very deep root system allows it to go without water for several months.

It has a very large number of vernacular names, including Ben tree, Winged Ben, neverdié, Anamambo, Horseradish tree, Drumstick tree, and also the tree that never dies or “miracle tree”. Indeed, it is known in ayurvedic medicine to cure 300 illnesses in addition to having a very substantial nutritional value. The fruits are eaten cooked, the leaves are consumed as a vegetable and have such nutritional value that they are a solution to malnutrition in certain countries.

An edible oil, rich in oleic acid, is derived from the seeds which are also used as flocculants to sanitize water. The oil is obtained by pressure or hexane extraction of the seeds whose tegument has been removed. For use of the flocculant properties, the oilseed cake is recovered after pressing.

The seeds can be eaten as beans when they are still green. Mature seeds contain about 40% oil. Oil from Moringa sp. is a good quality cooking oil (similar to olive oil) also used in perfumery, for the fabrication of soap or as lamp oil (it is very stable to oxidation).

In traditional medicine, the oil is used in application to ease pain during attacks of gout or rheumatism (The Indian Materia Medica, pp 811-816). Taken orally, the seeds are used as an antipyretic (Hukkeri et al., Indian J. Pharm. Sci. (2006) 68, pp. 124-126).

The oil from the seeds, obtained by pressing or by very non-polar extraction (with hexane, in particular) is widely used in cosmetology for its nourishing properties due to the triglycerides which it contains.

The use of an aqueous seed extract in cosmetology is described: the peptides and proteins which it contains would have anti-wrinkle and purifying properties: the seeds are first de-hulled and de-fatted (U.S. Pat. No. 6,500,470 and US 2006/0275247). The aqueous and de-fatted protein fractions extracted from whole or de-hulled seeds of Moringa sp. have hydrating, restoring, and anti-wrinkle effects on the skin (patent EP 1 064 008). Said protein fraction of seeds can be characterized as being “very polar”.

According to the literature, the seeds are composed of sterols (campesterol, stigmasterol, β-sitosterol, Δ5-avenasterol, clerosterol . . . ), fatty acids (C18:1-oleic-68 to 76%, C16:0 6 to 7.8%, C18:0 4 to 7.6%, C20:0 2.8 to 4% and C22:0 5 to 6.7%), proteins (26 to 32%), fibers, tocopherol (α, γ, δ: respectively 134, 93, and 48 mg/kg of oil).

The seeds also contain glucosinolates, including 4-(α-L-rhamnopyranosyloxy)-benzylglucosinolate. It has also been described that the leaves and seeds of the Moringa sp. contain some cytokinins such as zeatin, dihydrozeatin, and isopenthyladenine (patent US 2006/0222682).

The Applicant has demonstrated the use of a specific extract of whole seeds of Moringa sp. comprising oil (including triglycerides, fatty acids, and polar lipids) and of polyphenols as active principle in cosmetics and/or dermatological compositions.

Thus, the object of the present invention is an extract of whole seeds of Moringa sp. containing oil (including triglycerides, fatty acids, and polar lipids) and polyphenols. In the context of the present invention, “whole seeds” must be understood as seeds whose tegument has not be removed.

The extract of whole seeds of Moringa sp. is characterized by (% in weight compared to the weight of the dry extract):
an oil content of 5% to 50% including (i) 2% to 10% of triglycerides and fatty acids and (ii) 5% to 15% of polar lipids;
a content of total polyphenols of 0.01% to 5% (expressed as g of pyrogallol for 100 g of dry extract).

According to a characteristic of the invention, said extract has an oil content of 25% to 40% (% in weight compared to the weight of the dry extract).

According to another characteristic of the invention, the species of Moringa is preferably the Moringa oleifera or the Moringa drouhardii.

Said extract of Moringa sp. according to the present invention, for which cosmetic and dermatological valorizations have been demonstrated, is obtained from whole seeds of Moringa sp., advantageously dried, ground, then subjected to at least one extraction by a moderately polar solvent.

In the context of the present invention, the term [symbol] moderately polar solvent [symbol] must be understood as a solvent selected from among the group consisting of a C1 to C4 alcohol, acetone, a water/alcohol mixture, and a water/acetone mixture, used alone or combined.

Preferably, it will be an ethanol/water mixture. Advantageously, this ethanol/water mixture will be characterized by various proportions ethanol/water from 9/1 to 7/3 (v/v).

Even more advantageously, the moderately polar solvent is an ethanol/water mixture of 9/1 or 3/1 (v/v).

The extraction is carried out under agitation or under static conditions, with reflux or at ambient temperature, in a plant/volume ratio of solvent which can vary from 1/5 to 1/20 for a duration of 30 minutes to 48 hours. The extraction can be renewed 2 or 3 times.

The marc is then separated from the extract by centrifuging or filtration and the solution can be more or less concentrated until a dry extract with yield from 5% to 10% is obtained. The extract obtained not being very homogeneous, a support can be added during the drying step in mass proportions with respect to the extracted dry matter which can vary from 1% to 75%. The support can be maltodextrine, lactose, silica or any other cosmetologically acceptable support solubilizing the extract such as, for example, the propylene glycol/ethoxylated oleic alcohol in various proportions.

The Moringa sp. extract obtained is characterized by its oil (including triglycerides, fatty acids, and polar lipids) and polyphenol content.

Another object of the present invention relates to such an extract of Moringa sp. for use as an anti-aging active principle.

Preferably, said extract is adapted to fight all of the signs of cutaneous aging for people having a mature skin. In the context of the present invention, the term “mature skin” must be understood as the skin of people being conventionally over 55-years old, and preferably over 60-years old

The signs of aging of the skin are characterized, in particular, by a loss of firmness and/or elasticity and/or tonicity and/or suppleness of the skin and by the appearance of wrinkles and fine lines.

The object of the present invention is thus to provide a new active ingredient capable of providing at the same time protection, hydration, and nourishment to the epidermis/mature skin, and consequently to provide the skin with smoothing, anti-sagging, restructuring effects.

From different types of tests, the Applicant has evaluated the global anti-aging action of Moringa sp. extracts according to the present invention.

It has been shown that this new extract brings about at the same time the various desired actions, namely:
an antioxidant, anti-radical action to limit the oxidation process linked to intrinsic and extrinsic aging;
an action on the restoration of the barrier function of the skin (proteinic and lipid structure of the epidermis) altered with age: the use of said extract makes it possible to limit the cutaneous dehydration and thus protects the skin;
an action on the extracellular matrix to enhance the mechanical properties of mature skin (firmness, elasticity, tonicity).

The extract according to the present invention promotes the hydration, smoothing, non-sagging, and restructuration of the skin. The extract according to the present invention thus makes it possible to beautify and unify skin tone.

Another object of the present invention relates to the use of an extract such as defined to reinforce and restore the barrier function of the skin.

In the context of the invention, the term “reinforce the barrier function of the skin” means to improve the barrier function of the skin.

One of the fundamental functions of the skin is to ensure a barrier between the body and the outside environment, opposing itself, in one direction, to the penetration of the epidermis by fungi, bacteria, and allergens of the environment ( [symbol] outside-in [symbol] ) and in the other direction, to water loss ( [symbol] inside-out [symbol] ).

The epidermis is a pluristratified epithelium, of ectodermal origin, in constant renewal. Several layers of different morphological nature and cellular composition can be distinguished, from the inside out: the basal layer, cell layer whose keratinocytes have a very high proliferation capacity which allows for the autorenewal of the epidermis, the suprabasal layers (granular, spinous layers), and finally the horny layer (Stratum Corneum, SC). These stages correspond to more and more advanced levels of keratinocyte differentiation. The keratinocytes of the basal layer lose their proliferation capacity as soon as they start a process of migration toward the surface of the epidermis during which the keratinocytes express a differentiation program leading to cornification, a process of programmed cell death. The cutaneous barrier function is first carried out by the horny layer, a solid and sealed assembly made of two compartments:
an intercorneocytar cement rich in lipids: the lipids, organized in sheets and covalently connected to cells, limit the penetration of molecules through the horny layer;
layers of cornified, dead cells deprived of organites (corneocytes), which correspond to the final stage of keratinocyte differentiation.

The integrity of the extracellular lipid cement as well as of all the cellular elements of the horny layer and the balance between proliferation and keratinocyte differentiation are crucial to maintain a functional epidermal barrier function.

The perturbation of the barrier function, chronic or acute, makes the body sensitive to outer stress and to dehydration.

The improvement of the barrier function is in particular determining when the barrier function of the skin is altered and must be reestablished. This is the case in a certain number of physiological states, in response to time (cutaneous aging), or in connection with the hormonal context or stress. The speed of this restoration allowing the return to homeostasis is delayed. In addition, the cutaneous barrier function is altered in the majority of the most common pathologies of the skin in the population which are often accompanied with an inflammatory component (dryness of the skin . . . ).

Improving the barrier function can also be advantageous when the original function of the skin is to be consolidated, particularly in order to provide the body with better resistance to outside stress against which it can be exposed, particularly of the environmental type (ultraviolet rays, humidity level, outside temperature, pollution, burns). The barrier function of the skin comprises all the mechanisms of natural defense against stress to which it is subjected. The essential element carrying out this function is located in the most superficial portion of the epidermis, in the area of the horny layer referred to as stratum corneum.

It has been demonstrated from different tests that the extract such as defined advantageously has an action of restoration of the lipid structure and of the proteinic structure of the epidermis.

The use of the extract according to the present invention is particularly effective for the reinforcement and restoration of the cutaneous barrier function.

Another object of the present invention relates to a cosmetic and/or dermatological composition comprising, as an active principle, an extract of whole seeds of Moringa sp. according to the invention and at least one cosmetologically and/or dermatologically acceptable excipient.

Said cosmetic and/or dermatological composition according to the present invention comprises a quantity of dry extract of whole Moringa sp. seeds comprised between 0.1 g and 5 g for 100 g of said composition.

Advantageously, said quantity of Moringa sp. extract is comprised between 0.25 g and 1 g for 100 g of cosmetic and/or dermatological composition.

More particularly, the invention relates to an anti-aging cosmetic composition. Preferably, said cosmetic composition is adapted to fight all of the signs of cutaneous aging for people with mature skin.

The anti-aging cosmetic composition according to the present invention can further contain one or several active principles such as active ingredients adapted for solar protection and/or active ingredients adapted for skin depigmentation.

The active ingredients adapted for solar protection are further chosen from among chemical synthesis molecules known for their anti-UVA action, for their anti-UVB action such as octocrylene and/or dioctyl butamido triazone and/or bis-ethylhexyloxyphenyl methoxyphenyl triazine.

The active [ingredients] adapted to the depigmentation of the skin, to lighten and unify skin tone can be, in addition, niacinamide, vitamin C and its derivatives.

The cosmetologically acceptable excipient in view of obtaining an anti-aging cosmetic composition is chosen to enable a topical or oral administration.

Advantageously, the topical form is selected from among the group consisting of milk, cream, balm, oil, lotion, gel, foaming gel, pomade, spray, etc.

Advantageously, the oral form is selected from among the group consisting of tablets, capsules, lozenges, powder, granules, solutions or oral suspensions.

More particularly, the invention also relates to a cosmetic and/or dermatological composition adapted to strengthen and restore the barrier function of the skin.

The cosmetologically and/or dermatologically acceptable excipient in view of obtaining a cosmetic and/or dermatological composition strengthening and restoring the barrier function of the skin is chosen to enable a topical administration.

Advantageously, the topical form is selected from among the group consisting of milk, cream, balm, oil, lotion, gel, foaming gel, pomade, spray, etc.

Another object of the present invention relates to a cosmetic method to fight all the signs of cutaneous aging for people having a mature skin, characterized in that it involves the use, in topical or oral administration, of an extract of whole seeds of Moringa sp. according to the invention.

The preparations and compositions that follow are given by way of non-limiting examples.

### EXAMPLES OF PREPARATION OF THE PLANT EXTRACT

### Example 1

2.5 kg of whole seeds of Moringa oleifera, dried and ground, are extracted in 17.5 L of ethanol 90 (proportion ethanol/water=9/1) by two counter-current extractions at 80° C. After the environment has cooled at 50° C., the extracted solution is recovered by separation solid/liquid. The sample is dried allowing for obtaining 243 g of extract. This extract is characterized by an oil content of 36% including (i) 10% of triglycerides and fatty acids and (ii) 10% of polar lipids and a content of total polyphenols of 0.02% (expressed as g of pyrogallol for 100 g of dry extract).

### Example 2

20 g of whole seeds of Moringa oleifera, dried and ground, are reflux extracted in 100 ml of the ethanol/water mixture 75:25 for 1 hour. The extracted solution is recovered by separation solid/liquid and dried with a rotary evaporator at 50° C. 1.66 g of extract are thus obtained in the form of a brown paste titrating at 5% of oil and 0.68% of total polyphenols expressed as pyrogallol.

### Example 3

20 g of whole seeds of Moringa drouhardii, dried and ground, are reflux extracted in 200 ml of the ethanol/water 90:10 mixture for 1 hour. The extracted solution is recovered by solid/liquid separation and dried with a rotary evaporator at 50° C. 1.29 g of extract are thus obtained in the form of a yellow-brown paste titrating at 35% of oil (triglycerides, fatty acids, and polar lipids) and 1.3% of total polyphenols expressed as pyrogallol.

### Examples of Cosmetic Compositions

### Example 4

### Eye Care

| Compound | Quantity | |
|---|---|---|
| Dry extract Moringa sp. seed | 0.5 | g |
| Tocopheryl acetate (alpha) | 0.5 | g |
| Dextran sulfate | 0.3 | g |
| Dioctyl butamido triazone | 1 to 10 | g |
| Octocrilene | 1 to 10 | g |
| Bis-ethylhexyloxyphenyl methoxyphenyl triazine | 1 to 10 | g |
| Wax glucoside 202 | 1 to 5 | g |
| Stearate GLY./PEG-100 stearate | 1 to 5 | g |
| Benzoate C12-C15 | 5 | g |
| Pentanoate(neo)isodecyl | 1 to 8 | g |
| Siloxane(cytclopenta)decamethyl | 1 to 8 | g |
| Glycerol 99.5% | 1 to 5 | g |
| Hydroxyethyl acrylate/sodium acryloyldimethyltaurate | 1 | g |
| copolymer | | |
| Xanthan gum TF | 0.3 | g |
| Capryl glycol | sq | |
| Potassium sorbate | sq | |
| Purified water | sqp 100 | g |

### Example 5

### Brightening Revitalizing Cream

| Compound | Quantity | |
|---|---|---|
| Dry extract Moringa sp. seed | 0.5 | g |
| Tocopheryl acetate (alpha) | 0.1 | g |
| Niacinamide | 2 | g |
| Methoxycinnamate(p)ethylhexyl | 1 to 10 | g |
| Octocrilene | 1 to 10 | g |
| Bis-ethylhexyloxyphenyl methoxyphenyl triazine | 1 to 10 | g |
| Behenin(tri)/PEG-20 | 1 to 8 | g |
| Cetyl alcohol >95% | 1 | g |
| Palmitate glycol | 2 | g |
| Siloxane(cyclopenta)decamethyl | 1 to 5 | g |
| Methicone(di)200FL | 1 to 5 | g |
| Capric caprylic/trigly.30 70 | 1 to 5 | g |
| Methicone(cyclo)Mel.9040 | 1 to 5 | g |
| Xanthan gumTF | 0.3 | g |
| Hydroxyethyl acrylate/sodium acryloyldimethyltaurate | 0.7 | g |
| copolymer | | |
| Glycerol 99.5% | 1 to 5 | g |
| Capryl glycol | sq | |
| Sorbic acid | sq | |
| Butylhydroxytoluene | 0.01 | g |
| Titanium oxide/AI/Sericite Mel | 1 to 5 | g |
| Sodium hydroxyde | sq | |
| Purified water | sqp 100 | g |

Evaluation of the Antioxidant Activity

DPPH Test

The antioxidant activity of the seed extract of Moringa oleifera according to the present invention has been evaluated with the DPPH test. This test is based on the measure of the capacity for trapping antioxidants of the stable radical 2.2-diphenyl-1-picrylhydrazyl (DPPH). This stable radical, having absorption at 517 nm, is reduced to the corresponding hydrazine when it reacts with a hydrogen donor.

Results:

The results obtained are expressed in IC50, corresponding to the concentration giving 50% reduction in absorbance of a methanolic solution of DPPH at 0.06 mM.

| Product tested | IC₅₀ (μg/ml) |
|---|---|
| Non-polar extract (hexane) of whole seeds | >1 000 (inactive) |
| Seed extract devoid of tegument with EtOH 90% | 1 000 (inactive) |
| Extract according to Example 1 | 280 |
| Extract of tegument with EtOH 90% | 30 |
| Vitamin E (reference) | 6-10 |

The extract according to the invention has an antioxidant activity mostly provided by the molecules present in the tegument.

Chemiluminescence

It is a method which generates free radicals (superoxide radical O₂^{o-}) by a photochemical signal. The intensity of oxidation is 1000 times greater than that obtained under normal conditions. The detection is carried out by chemiluminescence and allows for the evaluation of antioxidant lipo- or hydrosoluble extracts or molecules. The results are expressed respectively in equivalent quantity of vitamin C or of Trolox (6-Hydroxy-2,5,7,8-tetramethylchroman-2-carboxylic Acid). The sensitivity is on the order of the nanomole.

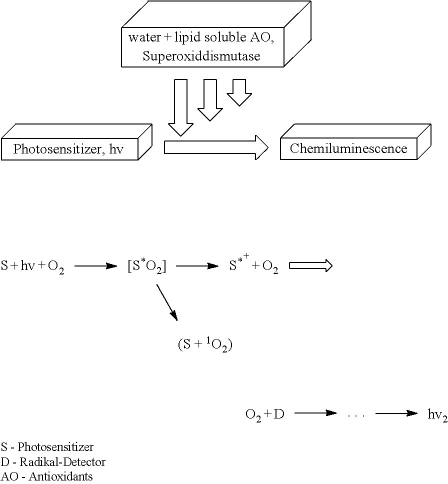

Results:

65 μg of extract according to Example 1 are necessary to obtain an activity equivalent to the activity detected for 1 μg of trolox: activity equivalent to lycopene, molecule known for its antioxidant activity.

This test has also confirmed that the antioxidant activity observed was mainly carried by the molecules of the tegument: only 4.7 μg of an extract EtOH 90% of tegument are necessary for 1 μg of trolox (activity equivalent to genistein). This confirms the importance of using, according to the invention, the whole seeds of Moringa sp.

The free radicals, whose production is increased due to outside stress (cold, pollution, tobacco, UV) are responsible for alterations of the DNA of the cutaneous cells, but also of the cell membranes and mitochondria. The anti-radical activity of the extract prepared according to Example 1 makes it possible to fight intrinsic and extrinsic cutaneous aging.

Evaluation of the Activity on the Restoration of the Barrier Function

The epidermis plays a major protective role by providing a chemical and mechanical barrier for the body. It ensures sealing is maintained, namely, the cutaneous barrier function. The corneocytes, keratinocytes of the horny layer, associated with a lipid matrix, ensure a great portion of this function. However, the deeper layers cooperate in the setting in place of the actors of this function. The differentiation capacity of the keratinocytes of the epidermis guarantee the setting in place of a barrier of the functional selective permeability type (Elias P M. J Invest Dermatol 125 183-200 (2005)).

From a proteinic standpoint, the epidermal differentiation is mainly centered on the evolution of structural proteins that are keratins and which contribute to the architectural integrity of the epidermis. Their expression varies as a function of the maturation degree of the keratinocytes. The base keratin 1 and the acid keratin 10 are early markers of keratinocyte differentiation, present from the basal layer of the epidermis. The expression of other markers of this biological process, later markers, can be followed such as that of the proteins of the corneous envelope, such as involucrin, as well as certain major enzymes at the origin of the cross-linking of the structural proteins to one another and with keratinocyte lipids, transglutaminases, such as transglutaminase 1 (TG1) (Houben E, et al. Skin Pharmacol Physiol.; 20(3):122-32 (2007)).

At the same time, the synthesis and the transport of keratinocyte lipids are at the origin of the inter-corneocyte lipidic cement indispensable to the cutaneous barrier, whose formation represents the last phase of the terminal epidermal differentiation. This extracellular lipid matrix provides the main barrier to transcutaneous movements of water and electrolytes (Mizutani Y., et al. FEBS Lett. 563: 93 (2004)). Thus, a certain number of enzymes and of lipid transporters have their keratinocyte expression augmented with the differentiation. In particular, certain members of the family of transporters ABC (Adenosine Triphosphate Binding Cassette transporter) play a major role in the steps of setting in place the lipidic barrier. Thus, ABC G1 which transports specifically the glycerol, and ABC Al2, indispensable to the transfer of the lipid precursors in the lamellar bodies, are sensitive markers. The epidermal ceramides play a major specific role and represent an essential marker of the level of functionality of the cutaneous barrier. Thus, the enzymes cooperating in the synthesis of the cutaneous ceramides have their expression and their activity specifically increased during the rupture of the epidermal barrier function and with the level of epidermal differentiation. This is, in particular, the case of the Sphingolipid C4-hydroxylase/delta-4 desaturase or hDES2 whose dihydroceramide hydroxylase activity contributes to the synthesis of the cutaneous phytoceramides in Man (Feingold, K. R. J Lipid Res 48: 2531-2546 (2007)).

First, the effects of extracts according to the invention have first been studied from a model of differentiated normal human keratinocytes (NHK).

This effect was then subsequently studied from a model of keratinocyte senescence.

Model of Differentiation of Normal Human Keratinocytes

The effect of the extracts of Moringa sp. on the gene expression of different proteins involved in the synthesis or the transport of the epidermal lipids, ABC G1 and hDES2, has been analyzed.

Results:

The results are expressed in percentage of stimulation of the gene expression (mRNA) of different markers of the epidermal differentiation expressed by the NHKs (with respect to the non-treated cells). A significant positive effect is considered from 100% of stimulation. The results obtained are presented in the table below.

TABLE 1
effect of vitamin D3, of roziglitazone, and of the extract
according to the invention from a model of differentiated
normal human keratinocytes (NHK).

| Cells treatments | ABC G1 | hDES2 |
|---|---|---|
| Vit D3 5 μM | 170% | 780% |
| Roziglitazone 10 μM | 330% | 190% |
| Extract Example 1 (20 μg/ml) | 150% | 100% |

The extract prepared according to Example 1 at 20 μg/ml results in the gene expression of hDES2 and ABC G1. The extract prepared according to Example 1 allows for restoring the lipid epidermal differentiation at the origin of the setting in place of the hydrophobic barrier allowing for limiting cutaneous dehydration, particularly encountered in mature skin.

Model of Keratinocyte Senescence

The regeneration capacities of the cutaneous barrier being reduced in mature subjects (Tagami, Arch. Derm. Res. 2007), we have used a model mimicking the keratinocyte senescence process from a lineage of human keratinocytes HaCaT treated with H₂O₂. We have analyzed the effect of the Moringa sp. extracts on the restoration of the expression (mRNAs) of the markers of proteinic differentiation whose expression was inhibited by the senescence of the cells, such as K1 and involucrine.

Results:

The extract according to Example 1, at 1 and 10 μg/ml allows for restoring the expression of K1 (11 and 35% respectively) and of the involucrine (15% for 10 μg/ml).

### CONCLUSION

The results obtained from the two models show well the complementarity of action (both on the restoration of the lipid structure and the proteinic structure of the epidermis) of the extract of the present invention.

Evaluation of the Activity of the Extracellular Matrix

The extracellular matrix (ECM) is a dynamic structure having a structural and regulating role for the tissues. It gives the skin its turgescence and mechanical properties: firmness, elasticity, and tonicity. In the area of the epidermis, it occupies the intercellular space and plays a role in maintaining the epidermal structure. It also provides the exchanges between the cells of the epidermis and plays a role in cell activity. The ECM of the epidermis is constituted, in particular, of collagen (fibrous protein) of the type IV. When a cell is senescent, the components of the ECM are predominantly degraded by enzymes of the zinc endopeptidases type called matrix metalloproteinases or MMPs. The latter actively participate in the scarring process but they also contribute to cutaneous sagging and to the appearance of wrinkles which are the first signs of cutaneous aging. Among them, the MMP-9 is a gelatinase which has an activity against denatured collagen molecules (gelatine) but can also cleave native molecules of collagen of the type IV, V, and VII.

The effect of Moringa sp. extracts on the expression of mRNAs of MMP-9 were analyzed on a lineage of human keratinocytes HaCaT treated with H₂O₂ mimicking the process of cell senescence.

Results:

The extract prepared according to Example 1 inhibits the gene expression of MMP-9 at 1, 10, and 30 μg/ml significantly at the three concentrations.

The extract prepared according to Example 1 makes it possible to restore the mechanical properties of the ECM: firmness, elasticity, and tonicity of the ECM of the skin and makes it also possible to restore the proteinic structure of the epidermis.

1-15. (canceled)

16. An extract of whole seeds of Moringa species comprising:
a) an oil content of 5% to 50% by weight of a dry Moringa species extract, wherein the oil content comprises 2% to 10% triglycerides and fatty acids, and 5% to 15% polar lipids; and
b) 0.01% to 5% total polyphenols expressed as grams of pyrogallol per 100 g of dry Moringa species extract.

17. The extract of whole seeds of Moringa species of claim 16, which is obtained by extraction using a moderately polar solvent.

18. The extract of whole seeds of Moringa species of claim 16, wherein the Moringa species is Moringa oleifera or Moringa drouhardii.

19. A method for preparing the extract of whole seeds of Moringa species of claim 16, comprising the following steps:
a) grinding of the whole seeds;
b) extracting at least once with a moderately polar solvent;
c) centrifuging or filtering; and
d) drying.

20. The method of claim 19, wherein the moderately polar solvent is selected from a C1 to C4 alcohol, acetone, a water/alcohol mixture, a water/acetone mixture, and combinations thereof.

21. The method of claim 19, wherein the moderately polar solvent is an ethanol/water mixture.

22. The method of claim 21, wherein the ethanol/water proportion is between 9/1 and 7/3 (v/v).

23. A method of treating cutaneous aging comprising the step of administering the extract of whole seeds of Moringa species of claim 16 to a subject exhibiting cutaneous aging and/or mature skin.

24. The method of claim 23, wherein the extract of whole seeds of Moringa species is adapted to treat all signs of cutaneous aging in subjects having mature skin.

25. The method of claim 23, wherein the administration of the extract of whole seeds of Moringa species strengthens and restores the barrier function of skin.

26. The method of claim 23, wherein the extract of whole seeds of Moringa species is administered topically to skin and/or orally to a subject exhibiting cutaneous aging and/or mature skin.

27. A cosmetic and/or dermatological composition comprising as active principle, the extract of whole seeds of Moringa species of claim 16 and at least one cosmetologically and/or dermatologically acceptable excipient.

28. The composition of claim 27, wherein a quantity of dry extract of whole seeds of Moringa species is between 0.1 g and 5 g per 100 g of the composition.

29. The composition of claim 27, which is an anti-aging cosmetic composition which is adapted to treat all of the signs of cutaneous aging in subjects having mature skin.

30. The composition of claim 29, further comprising one or more active principles for solar protection and/or active ingredients for skin depigmentation.

31. The composition of claim 27, which strengthens and restores the barrier function of skin.
